(19) United States
(12) Patent Application Publication
(10) Pub. No.: US 2008/0233003 A1
(43) Pub. Date: Sep. 25, 2008

(54) SYSTEM AND METHOD FOR TREATING LIQUID USING A CORONA DISCHARGE PROCESS IN A LOW PRESSURE ENVIRONMENT

(76) Inventor: Yakov E. KRASIK, Nesher (IL)

Correspondence Address: Pearl Cohen Zedek Latzer, LLP, 1500 Broadway, 12th Floor, New York, NY, 10036, US

(21) Appl. No.: 12/053,867

(22) Filed: Mar. 24, 2008

Related U.S. Application Data
(60) Provisional application No. 60/907,143, filed on Mar. 22, 2007.
(60) Provisional application No. 60/907,144, filed on Mar. 22, 2007.

(51) Int. Cl. A61L 2/03

(52) U.S. Cl. 422/22; 422/186.04

(57) ABSTRACT

A method for oxidizing and disinfecting liquids including maintaining a reactor under sub-atmospheric pressure conditions; passing liquid within the reactor over a first electrode; and generating a high voltage, short pulse to a second electrode located opposite the first electrode, over the liquid that passes over the first electrode to create corona discharge. A reactor and an electrode suitable for use in such reactor are also disclosed.

### CROSS REFERENCE TO RELATED APPLICATIONS

This application claims priority from U.S. Provisional Patent No. 60/907,143, filed on Mar. 22, 2007, and U.S. Provisional Patent No. 60/907,144, filed on Mar. 22, 2007 both of which are incorporated in their entirety herein by reference.

### FIELD OF THE INVENTION

The present invention relates to corona discharge initiation reactions. More specifically, the present invention relates to a system and method for treating liquids via corona discharge initiation reactions.

### BACKGROUND OF THE INVENTION

Initiation of a corona discharge may be brought about by applying a high voltage pulse to an electrode, in the neighborhood of another electrode. When the applied voltage has a large enough negative amplitude, the electric field at the surface of the first, high voltage electrode will exceed the threshold, E_{cr}, necessary to initiate a corona discharge.

Depending on the polarity of the high-voltage electrode, either a positive or a negative corona discharge will be generated (Yu P. Raizer, 1987). Assuming the voltage is applied to the electrode with the greater curvature, either type of discharge results in a high density plasma formation in the vicinity of the high-voltage electrode having the stronger curvature, with the density decreasing rapidly towards the second electrode having a smaller curvature.

In the case of point-plane configuration, where the high-voltage electrode is needle-shaped and the second electrode is planar, one can use the approximation: E≈V/5r, as a rough estimate for the electric field E at the point apex, where V is the potential difference between the point apex and the plane.

The total current of the corona discharge consists of electron current and ion current flowing towards the anode and cathode respectively. The ions are created as a result of background gas ionization by fast electrons. The corona current consists of electrons emitted by the cathode, ion current moving towards the negative electrode and secondary electrons which are created as a result of ionization and ion bombardment of the cathode.

The discharge current and the threshold electric field greatly depend on the curvature of the electrode which can be decreased significantly during the electrode operation due to surface erosion and melting (melting decreases significantly the corona discharge current).

The corona discharge has a number of commercial and industrial applications. One of the known applications of corona reactions is the production of oxidizing agents such as ozone (O₃) and hydroxide (OH) radicals which depend on the parameters of the background gases and on the corona discharge.

Ozone has many industrial applications such as, for example, treating of liquids (i.e., oxidizing liquids).

Oxidation processes for treating liquids are used in many fields and industries, such as, for example, the petrochemical industry, refineries, the pharmaceutical industry, the chemical industry, the textile industry, the Pulp & Paper industry, water disinfection, the pesticide industry and many more.

Typical uses of oxidation processes may be in applications such as pretreatment for biological wastewater treatment, wastewater polishing, ground water remediation to treat MTBE (methyl tertiary butyl ether), Dioxane, Trichloro ethylene, perchloro ethylene, and other organo-halogens, landfill leachate treatment, cooling tower water reuse, storm water run-off treatment, and onboard wastewater treatment (in marine vessels).

As noted above, one of the known applications of corona reactions is the production of oxidizing agents such as ozone (O₃) for oxidizing liquids. As oxidation processes require relatively high concentrations of ozone and other free radicals, various ways to optimize such processes are currently available. These include an approach which involves the application of a more intense corona discharge, and an approach which involves increasing the oxygen concentration.

However, both approaches suffer from drawbacks e.g., applying a more intense corona discharge shortens the lifetime of the corona's electrode and increasing the oxygen concentration requires an additional oxygen supply.

In addition, when producing ozone via corona reactions under atmospheric pressure, the ozone concentration decreases rapidly as the distance from the electrode increases.

An aim of the present invention is to provide a robust system and method for treating liquids employing corona discharge initiation reactions.

Further features of the proposed system and method for liquid treatments according to preferred embodiments of the present invention will be explained in detail below with reference to the attached drawings.

### SUMMARY OF THE INVENTION

There is thus provided, according to embodiments of the present invention a reactor for treating liquids comprising:

a container having an electrically conductive open channel serving as a first electrode;

a second electrode positioned opposite the first electrode;

a high voltage generator coupled to said second electrode; and

vacuum pump for generating sub-atmospheric pressure conditions within the container.

Furthermore, in accordance with embodiments of the present invention, the first electrode is made from a metallic material.

Furthermore, in accordance with embodiments of the present invention, the second electrode is comprises a plurality of fiber bundles, each of the fiber bundles comprising a plurality of individual fibers of different lengths with respect to an operation end of the electrode device and a screening ring around the fiber bundles.

Furthermore, in accordance with embodiments of the present invention, there is provided a method for oxidizing and disinfecting liquids comprising:

maintaining a reactor under sub-atmospheric pressure conditions;

passing liquid within the reactor over a first electrode; and

generating a high voltage, short pulse to a second electrode located opposite the first electrode, over the liquid that passes over the first electrode to create corona discharge.

Furthermore, in accordance with embodiments of the present invention, the second electrode is comprises a plurality of fiber bundles, each of the fiber bundles comprising a plurality of individual fibers of different lengths with respect to an operation end of the electrode device and a screening ring around the fiber bundles.

Furthermore, in accordance with embodiments of the present invention, the reactor is maintained at sub-atmospheric pressure conditions of about 0.02-0.08 Bar.

Furthermore, in accordance with embodiments of the present invention, the method further comprises post treating of the liquid and dispensing of the treated liquid from the container.

### BRIEF DESCRIPTION OF THE DRAWINGS

In order to better understand the present invention, and appreciate its practical applications, the following figures are provided and referenced hereafter. It should be noted that the figures are given as examples only and in no way limit the scope of the invention. Like components are denoted by like reference numerals.

FIG. 1 is a top-view schematic illustration of a microstructured fiber corona discharge electrode in accordance with embodiments of the present invention.

FIG. 2 is a cross sectional side-view of a microstructured fiber corona discharge electrode in accordance with embodiments of the present invention.

FIG. 3 is a schematic illustration of a corona discharge electrode according to embodiments of the present invention, showing the plasma region created in proximity to the edges of the fibers.

FIG. 4 is a block diagram that illustrates a method for treating liquids via corona discharge initiation reactions in a reactor operated under atmospheric pressure conditions in accordance with embodiments of the present invention.

FIG. 5 illustrates a reactor for water treatment under atmospheric conditions employing a corona discharge electrode according to embodiments of the present invention.

### DETAILED DESCRIPTION OF PREFERRED EMBODIMENTS

In the following detailed description, numerous specific details are set forth in order to provide a thorough understanding of the invention. However it will be understood by those of ordinary skill in the art that the present invention may be practiced without these specific details. In other instances, well-known methods, procedures, components and circuits have not been described in detail so as not to obscure the present invention.

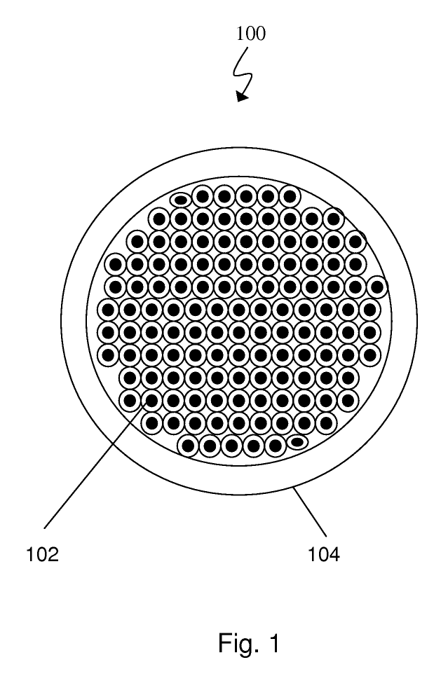

Referring now to FIG. 1, microstructured fiber corona discharge electrode 100 may include one or more (typically a plurality of) fiber bundles 102, which are typically made of carbon fibers (or microtubes) in accordance with embodiments of the present invention. However, the use of other materials for the manufacturing of bundles of fibers other than carbon may be possible.

Fiber bundles 102 may be surrounded by a screening ring 104 as seen in the figure, electrically connected to the surface of either an anode or a cathode holder.

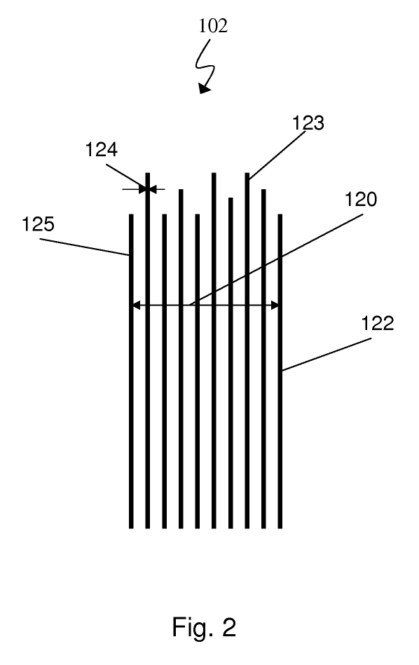

Referring now to FIG. 2, each fiber bundle 102 may have a diameter 120 typically of a few millimeters and may include many single fibers (or microtubes) 122 each having a diameter 124 typically of a few micrometers.

As seen in the figure, individual fibers 122 do not possess the exact same length at the operation end of the electrode device (the end that faces the opposite electrode). Therefore, as the electric field is stronger over the tips of the longest fibers 123 initially the latter participate in the production of corona discharge initiation reaction.

During the corona discharge operation, the emitting fibers may lose height and recede, allowing other fibers to participate in the corona discharge. Considering that each bundle typically includes several thousands of individual fibers, the operation life time of such an electrode is substantially longer than the operation life time of a needle electrode.

It should be noted that the sharp edges of the fibers generate a relatively strong electric field which may cause an intense corona discharge that is greater than the corona discharge created by the conventional electrode emitters which are commonly used.

Thus, a microstructured fiber electrode according to embodiments of the present invention is superior to the conventional electrode for corona discharge as it provides an intense corona discharge and possesses a longer life-time than the life time of the conventional electrode.

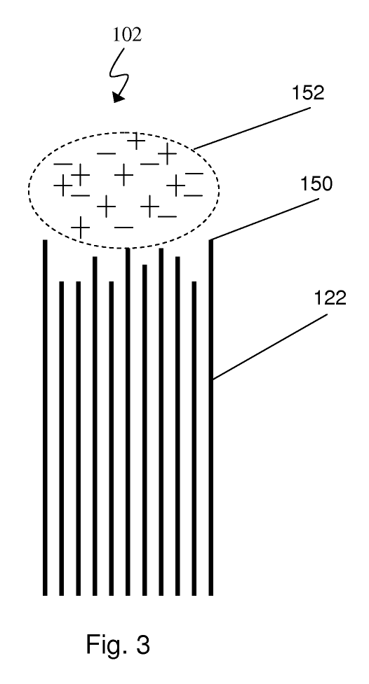

Referring now to FIG. 3, a strong electric field at the top edges 150 of fibers 122 leads to the creation of a plasma region 152.

As noted earlier, the corona discharge has numerous commercial and industrial applications as corona discharge electrodes can be used in “volume discharge” processes.

A “volume discharge” process is based on the development of electron streamers in a gas positioned between two electrodes. In this process, electrons, emitted from sharp edges or other electric field concentration points on the surface of the electrode coupled to the voltage source, gain on their “free run” path before collision enough energy to ionize a gas atom, producing at least one (but typically more than one) new electron that accelerates in the direction of the ground electrode.

The process is repeated many times, leaving an ionized path. This is a substantially high-impedance process, and a large number of streamers are developed simultaneously.

If the voltage pulse duration is shorter or comparable with the streamer development time, an arc stage discharge is not reached, and thus, heat is not created.

According to embodiments of the present invention an optimized system and method for treating liquids is disclosed, which overcomes the drawbacks of the above mentioned approaches. Water treatment is discussed, by way of example, but the present invention is not limited to water treatment alone and other liquids can be treated too, using a method and system according to embodiments of the present invention.

In accordance with some embodiments of the present invention, water is boiled in-situ to entail an intense evaporation of water molecules.

The evaporated molecules serve as an additional supply of oxygen, and thus, optimize the oxidation process.

It is known that the boiling point of water is around room temperature (297K) at a pressure of 0.04 Bar. Thus, in order to induce boiling of the water at room temperature, the reactor is operated under sub-atmospheric pressure conditions (pressure of approximately 0.04 Bar).

Treating liquids using a corona discharge initiation reaction is a “volume discharge” type process which is based on the development of electron streamers in the gas positioned between two electrodes.

In “volume discharge” type processes electrons, emitted from sharp edges or other field concentration points on the surface of the electrode coupled to the voltage source, gain on their “free run” path before collision enough energy to ionize a gas atom, producing at least one (or more) new electrons that start to move in the direction of the ground electrode.

The process is repeated many times, leaving an ionized path. This is substantially a high-impedance process, and a large number of streamers are developed simultaneously, and if the voltage pulse duration is shorter or comparable with the streamer development time, an arc stage discharge is not reached, and thus, heat is not created.

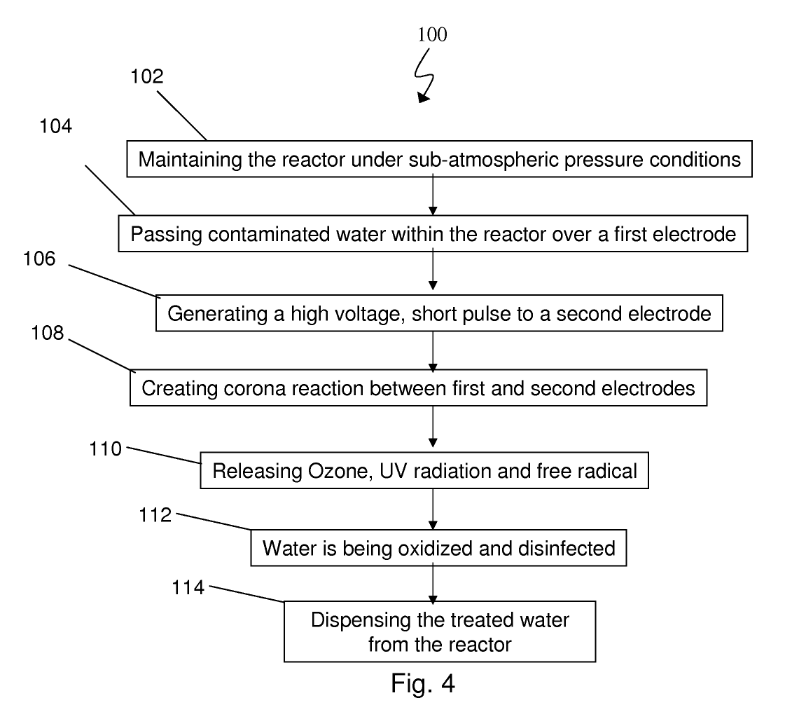

Referring now to FIG. 4, block diagram 100 illustrates a method for treating water via corona discharge initiation reaction in a reactor operated under low pressure conditions (e.g., at a pressure of approximately 0.04 Bar). As seen in the figure, the method comprises: maintaining a reactor under sub-atmospheric pressure conditions 102, passing contaminated water within the reactor over a first electrode 104, generating a high voltage, short pulse to a second electrode 106, creating corona reaction between first and second electrodes 108, releasing ozone, UV radiation and free radicals 110 (this is a result of the corona discharge), water is being oxidized and disinfected 112 and dispensing treated water from the reactor 112.

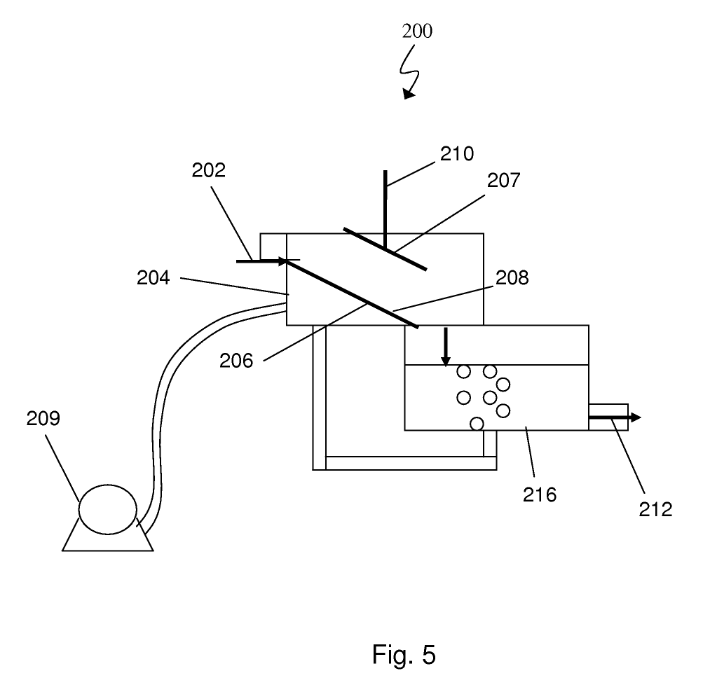

Referring now to FIG. 5, reactor 200 is utilized for treating water in a sub-atmospheric pressure conditions. As seen in the figure, contaminated water 202 flows within container 204 having a metallic open channel serving as a first electrode 206. A second electrode 207, is positioned above water surface 208.

Vacuum pump 209 is used for creating a pressure of about 0.04 Bar in reactor 200. However, other suitable vacuum means can be used to create a low pressure environment in reactor 200.

Upon generation of high voltage pulses from a high voltage, short pulse generator 210 that is coupled to second electrode 207, an electric discharge between second electrode 207 and water surface 208 is generated causing a corona reaction that releases ozone, UV radiation and free radicals which oxidize and disinfect contaminated water 202.

Post treatment, treated water 212 flows through and exits container 216.

An electrode, such as the one described in FIGS. 1-3, can be used as the second electrode in a reactor according to embodiments of the present inventions.

It should be noted that besides increasing the efficiency of the oxidation process, there are other advantages associated with treating water in a low pressure environment.

Namely, the value of the electric field necessary to initiate the corona discharge is significantly decreased, and the active zone of the corona is significantly increased with high energy electrons due to the increase in the mean free path of electrons.

Furthermore, the concentration of ozone and radicals may be increased not only due to an intense evaporation of water molecules but also due to an increase in their lifetimes.

In addition, an increase in the diffusion rate of ozone and radicals inside the water can also be expected in a low pressure environment due to water turbulence at temperatures close to the boiling temperature.

Finally, as the mean free path of electrons becomes comparable to the inter-electrode gap, direct interaction of corona electrons with water substance should be considered as well.

What is claimed is:

1. A reactor for treating liquids comprising:
a container having an electrically conductive open channel serving as a first electrode;
a second electrode positioned opposite the first electrode;
a high voltage generator coupled to said second electrode; and
vacuum pump for generating sub-atmospheric pressure conditions within the container;

2. The reactor of claim 1, wherein said first electrode is made from a metallic material.

3. The reactor of claim 1, wherein said second electrode is comprises a plurality of fiber bundles, each of the fiber bundles comprising a plurality of individual fibers of different lengths with respect to an operation end of the electrode device and a screening ring around the fiber bundles.

4. A method for oxidizing and disinfecting liquids comprising:
maintaining a reactor under sub-atmospheric pressure conditions;
passing liquid within the reactor over a first electrode; and
generating a high voltage short pulse to a second electrode located opposite the first electrode over the liquid that passes over the first electrode to create corona discharge.

5. The method as claimed in claim 4, wherein said second electrode is comprises a plurality of fiber bundles, each of the fiber bundles comprising a plurality of individual fibers of different lengths with respect to an operation end of the electrode device and a screening ring around the fiber bundles.

6. The method as claimed in claim 4, wherein the reactor is maintained at sub-atmospheric pressure conditions of about 0.04-0.08 Bar.

7. The method as claimed in claim 4, further comprising post treating of the liquid and dispensing of the treated liquid from the container.
